(19) United States
(12) Patent Application Publication
(10) Pub. No.: US 2017/0112983 A1
(43) Pub. Date: Apr. 27, 2017

(54) MASSAGING LACTATION ASSISTIVE DEVICE

(71) Applicant: Regents of the University of Minnesota, Minneapolis, MN (US)

(72) Inventors: Meghan Leigh Thorne, Minneapolis, MN (US); Catherine E. Taylor, Minneapolis, MN (US); Jack B. Stubbs, Orlando, FL (US); Benjamin Y. Arcand, Minneapolis, MN (US)

(21) Appl. No.: 15/331,235

(22) Filed: Oct. 21, 2016

Related U.S. Application Data
(60) Provisional application No. 62/244,982, filed on Oct. 22, 2015.
(60) Provisional application No. 62/244,955, filed on Oct. 22, 2015.

(51) Int. Cl. A61M 1/06; A61H 9/00; A61N 1/32; A61N 1/36; A61M 1/00; A61N 1/04

(52) U.S. Cl. CPC A61M 1/06; A61M 1/0019; A61M 1/005; A61N 1/0456; A61N 1/322; A61N 1/36014; A61N 1/0476; A61H 9/005; A61H 9/0071; A61H 2205/082

(57) ABSTRACT

Disclosed are lactation assistive devices. The lactation assistive device may include a stimulation component and a controller. The stimulation component may include electrical components or mechanical components to stimulate a breast. The stimulation component may be sized to fit within a garment worn about the breast and may define an opening to be located proximate a nipple of the breast when the garment is worn. The controller may be configured to control stimulation of the breast by the stimulation component.

### CLAIM OF PRIORITY

This patent application claims the benefit of priority of U.S. Provisional Patent Application Ser. No. 62/244,982, filed on Oct. 22, 2015, and the benefit of priority of U.S. Provisional Patent Application Ser. No. 62/244,955, filed on Oct. 22, 2015, each of which are hereby incorporated by reference herein in their entirety.

### BACKGROUND

Currently, lactating mothers may breastfeed directly from the breast or utilize breast pumping, formula, or any combination thereof to feed a child. There are many variables that can be considered in determining the best care for the mother and baby. The variables may include the health and strength of the baby, the health of the mother, the ability for the mother to build and maintain her milk supply, and whether the mother is comfortable enough to breastfeed or express milk when required.

### DESCRIPTION OF THE FIGURES

FIG. 1A shows a breast pump that uses vacuum through rigid cones to facilitate extraction of milk from a breast;

FIG. 1B shows use of a double pumping breast pump;

FIG. 2A shows a schematic of different sensory nerve fibers within a nerve bundle;

FIG. 2B shows different nerve stimulation pulses that may excite beta and delta fibers;

FIGS. 3A and 3B show an example electrode array inserts consistent with embodiments disclosed herein;

FIG. 3C shows a nursing bra with an electrode array insert with optional bra opening cover consistent with embodiments disclosed herein;

FIGS. 4A and 4B show examples of two electrode stimulation configurations:

FIG. 5 shows an example of a three electrode stimulation configuration;

FIGS. 6A-6D show electrode patterns for various electrodes;

FIG. 7A shows an example flexible collection container consistent with embodiments disclosed herein;

FIG. 7B shows an example stand-up container consistent with embodiments disclosed herein;

FIGS. 8A, 8B. 8C, and 8D show a collection device and a flexible container consistent with embodiments disclosed herein;

FIGS. 9A, 9B, and 9C show a schematic of multiple flexible compartments arranged circumferentially about a nipple consistent with embodiment disclosed herein;

FIGS. 10A, 10B, 10C, and 10D show an example of a soft actuator configuration and self-adhesive backing consistent with embodiments disclosed herein;

FIG. 11 shows an example of a fluid bladder system for mechanical stimulation;

FIG. 12 shows an example of a cable configuration of mechanical stimulation;

FIG. 13 shows an example of a bladder covered in fibers;

FIG. 14 shows an example of a bladder partially covered in fibers;

FIGS. 15A and 15B show an example of multiple bladders in a circumferential pattern;

FIGS. 16A, 16B, and 16C show an example of a multiple bladders in a radial pattern;

FIGS. 17A and 17B show examples of multiple bladders arranged in a crossed configurations;

FIGS. 18A, 18B, and 18C show an example of a circular bladder;

FIGS. 19A and 19B shows an example of a solid members and bladders used for stimulation; and

FIGS. 20A and 20B shows an example of articulating bladders used for stimulation.

### DETAILED DESCRIPTION

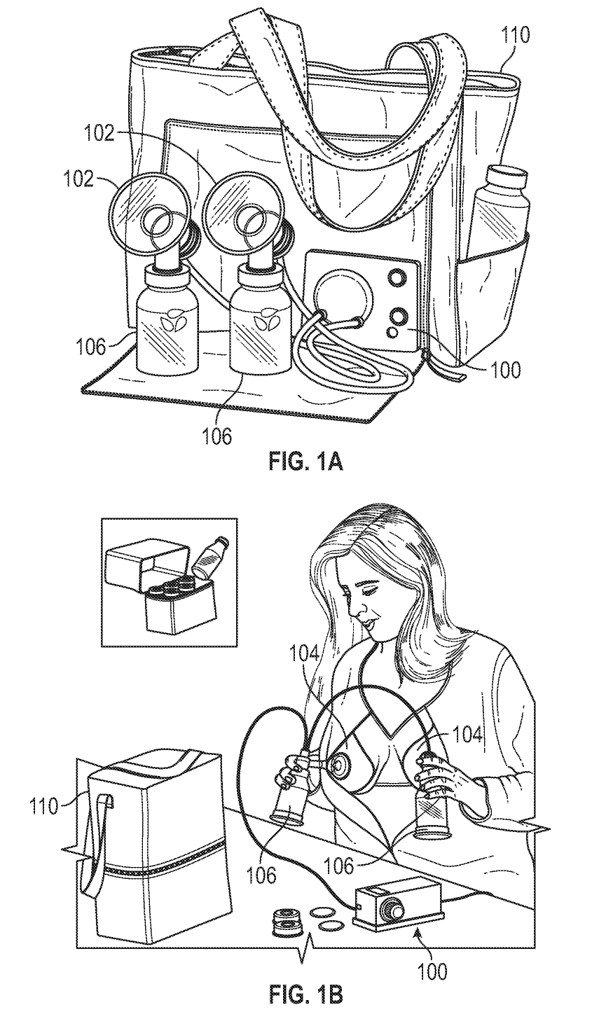

A hands-free and convenient breast pump/garment for mothers may be used to allow mothers to express breast milk at work or at home with decreased disruption of their daily activities. FIGS. 1A and 1B shows a conventional breast pump 100 that uses suction to express breast milk. As shown in FIGS. 1A and 1B, rigid conical cones 102 may be placed over the nipples 104. As suction is applied, milk may be expressed through the cones 102 and collected in bottles 106.

Mothers who return to work and use current breast pumps, such as those shown in FIGS. 1A and 1B, may face several barriers. These barriers may include disrobing at work (as shown in FIG. 1B, scheduling pumping, feeling embarrassed by the noise emitted by the pump, and feeling guilty for taking breaks. Breast pumping may require the mother to carry a cumbersome breast pump into a designated room, take equipment out of a storage bag 110 and assemble the parts, plug the pump into an outlet, disrobe, place breast shields over the nipples and hold them there while expressing milk. When completed, the mother may need to carefully remove the breast shields to not lose milk, clean all the parts, put everything back into the bag, put her clothes back on, and find a place to store the milk. Accomplishing all of this may take 30 minutes. If the mother does not express her milk, she may lose the milk supply. Once the milk supply starts to dwindle it may drastically fall off and mothers may have to turn to formula.

Moving away from cyclic suction of the mother's nipples into an obtrusive, rigid funnel may be pursued. Other ways to stimulate and induce the milk ejection reflex include, but are not limited to, hand expression. Hand expression is a technique that may be used, without a breast pump, to express milk when a baby may be sleeping, if the baby is premature and in the neonatal intensive care unit (NICU), and when mother and baby are separated. It has been found that massaging, as opposed to suction with a breast pump, may empty more of the breast milk from the breasts.

As disclosed herein, tactile stimulation of mammary and nipple tissue may facilitate both prolactin and oxytocin responses for milk expression. Tactile stimulation of mammary and nipple tissue, along with the use of simultaneous pumping and breast massage, may improve both milk volume and fat content. Women may feel strongly that expression without preconditioning by massage may be more difficult, and many may be reluctant to drop the method.

Tactile stimulation, as disclosed herein, along with simultaneous pumping and breast massage may also lead to an increase in the quality of breast milk by increasing the total solids, lipids, and casein concentration and gross energy. Therefore, not only can the amount of milk production be increased, but the quality of the milk may also be increased. Finally, breast massage techniques also may be used to alleviate complications including, but not limited to, engorgement, plugged ducts, and mastitis.

The embodiments disclosed herein may use massage for the stimulation of the breast while providing a low-profile, hands-free, convenient, and quiet design that may allow for easier milk expression and collection in semi-private or workplace scenarios. In addition, the embodiments disclosed herein may allow mothers to express and collect breast milk wherever they feel comfortable, at a location they may find convenient, and at what time may be best for them.

As disclosed herein, a lactation assistive device may induce let-down through massage via electrical and/or mechanical stimulation and may be efficient in assembly and cleanup, convenient and hands-free, and can fit multiple breast sizes.

As disclosed herein, a garment may include an electrode array that may be connected to a small electrical stimulation unit. The electrical stimulation unit may electrically stimulate breast tissue for the purpose of milk expression. The electrode array may be removable so the garment can be cleaned. In addition, the electrode array inserts may be made of a material that may promote direct contact with breast tissue. The electrode array may be a dry electrode array or contact the skin of a user without the use of conductive gels or other filler material.

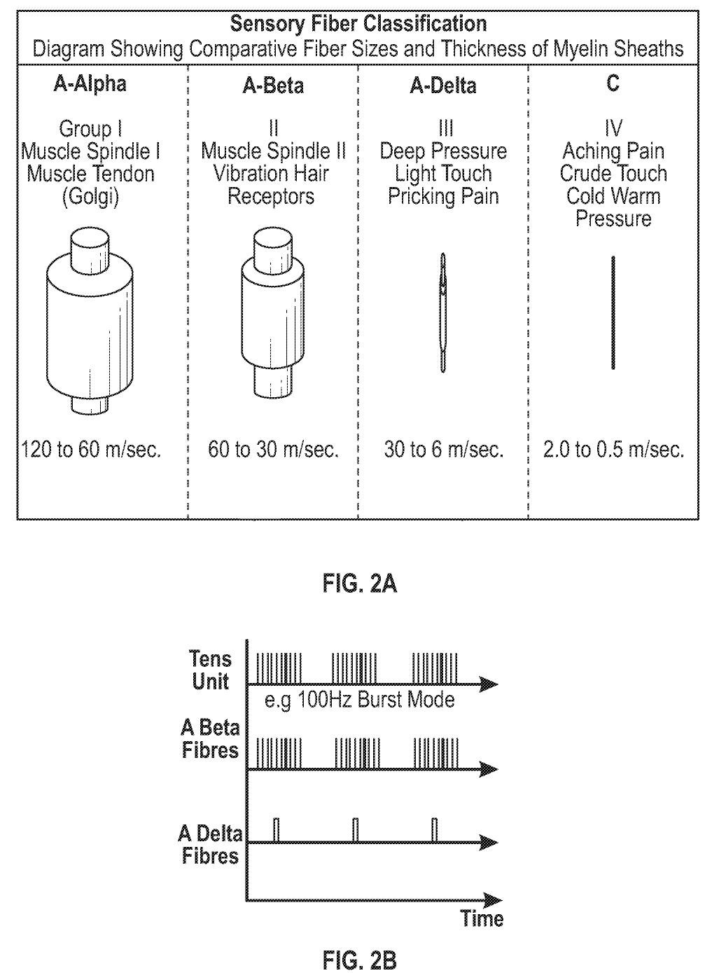

Electrical stimulation using an electrical stimulation unit, such as a Transcutaneous Electrical Nerve Stimulation (TENS) device, can induce oxytocin release and may promote milk let-down, even for mothers who are not currently lactating. One aim for electrical stimulation unit may be to provide a degree of symptomatic pain relief by exciting sensory nerves and electrically stimulating either the pain gate mechanism and/or the opioid system. The stimulation parameters for electrical stimulation units, such as TENS units, may include, but are not limited to, output intensity (0-80 mA); pulse frequency (2-150 Hz); and pulse width (50-250 μs). Stimulation of different nerve fibers, for example and as shown in FIG. 2A, may be possible by applying different pulse waveforms. Electrically stimulating the myelinated beta fibers through low intensity and high frequencies may create a sensation of touch. Stimulating the stronger and slower delta fibers through high intensity low frequency may stimulate muscle contraction. It may be possible to stimulate both fiber types by sending an electrical signal that may include both waveforms as shown in FIG. 2B.

With regards to the electrical stimulation units, the units, or controllers, can have safety limits to prevent harm to the user. For example, the controllers can have pre-set waveforms to select from, as well as the capability for the user to adjust or set (and save) her own custom waveforms. This would allow a user to select and save waveforms that works best for her.

In the case of nerves of the lactation system, electrically stimulating the beta and delta fibers may create a physical sensation that may simulate touching and massaging. Lactation consultants may encourage mothers to touch/comb/shake/vibrate, as well as hand massage their breasts and nipples to induce an oxytocin release and promote the milk ejection reflex (let-down).

Increased levels of hormones associated with lactation may be demonstrated with the electrical stimulation as disclosed herein. Targets of electrical stimulation may include, but are not limited to, mammary nerves, Vagal nerves, and neurohypophysis/pituitary.

Microcurrent stimulators, which may be similar to TENS but may use electrical currents that may be much lower in intensity, for example, on the order of 10⁻⁶ amps, may also be used for electrical stimulation. Microcurrent stimulation may also be used for skin rejuvenation and may improve skin tone. Microcurrent stimulation may not cause muscle contraction directly, microcurrent stimulation may increase an amount of adenosine triphosphate (ATP) within cells of a muscle by 300-500%. The ATP release may facilitate myoepithelial cell contraction in milk-ejection responses.

As disclosed herein, electrical stimulation of breast tissue may be used to stimulate afferent sensory nerves near the nipple, which may send afferent signals to the brain that may promote oxytocin release in the hypothalamus.

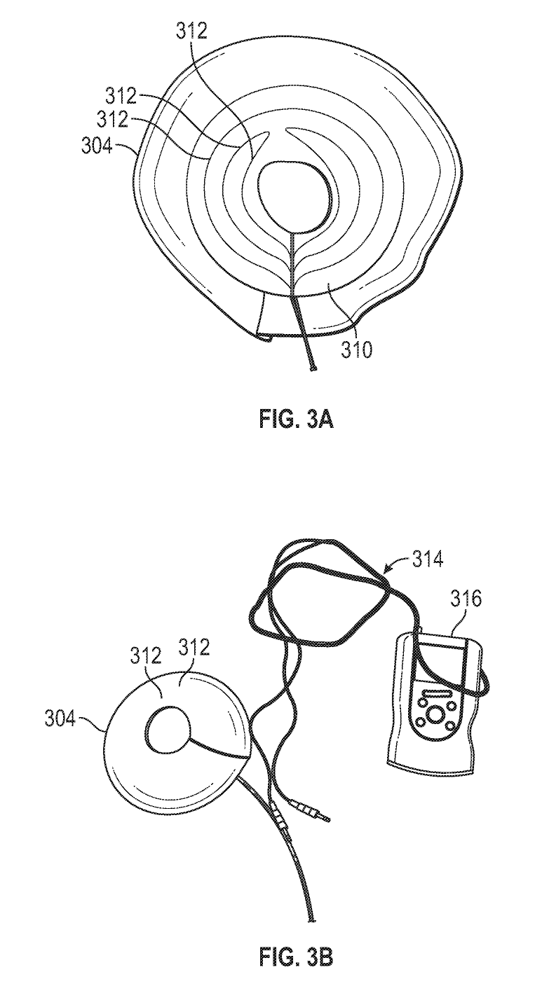

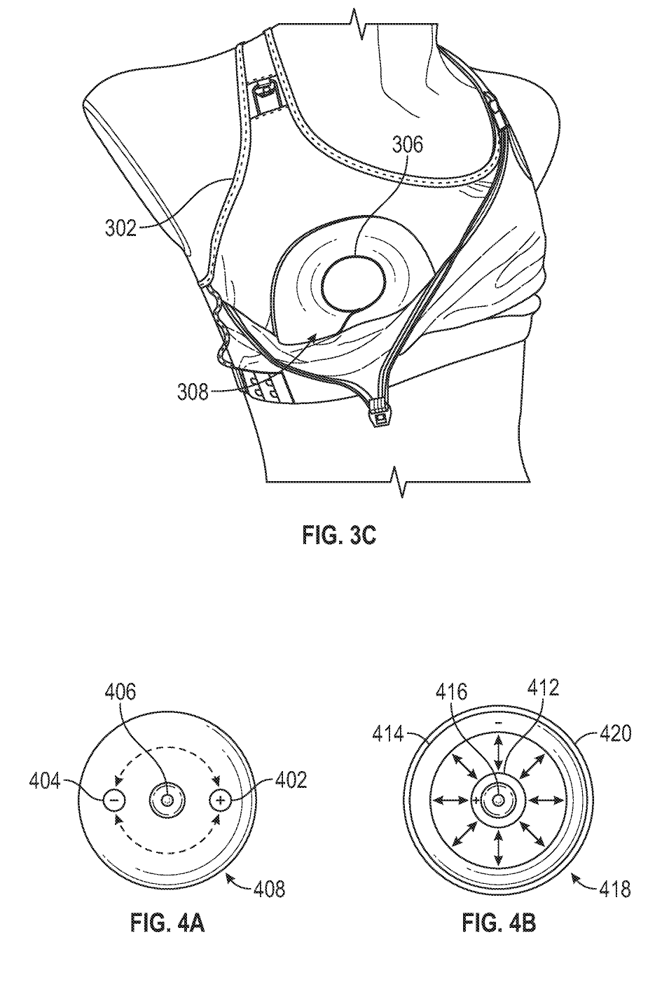

As shown in FIGS. 3A-3C, a bra-like garment 302 may be configured to receive electrode array inserts 304. The garment 302 may be adjustable to fit different breast sizes. The garment may include an opening 306 proximate the nipple 308 to allow for the connection of milk collection containers. The garment 302 may be close or otherwise cover the opening 306 at times when not expressing milk. The garment 302 may be worn with or without inserts 304. The inserts 304 can be placed within the garment 302 while wearing or not wearing the garment 302.

The electrode array inserts 304 may be made of a material that may promote direct contact with the breast tissue and may be held in place by the garment 302. Inserts 304 may be a shallow cone shape and may include an open end 310 that may adjust to different breast sizes.

The array of electrodes of inserts 304 may encompass each breast and may each consist of one or more electrodes 312. Electrical stimulation may be induced uniformly across the breast or with different stimulation parameters/waveforms and/or timing for different electrodes. For example, stimulation across the breast may include stimulating the breast tissue further away from the nipple 308 and progressing stimulation towards the nipple 308 over a fixed or varying time parameter. Another example may include changing stimulation parameters and patterns so that stimulation may feel better to the woman. In addition, switching stimulation parameters and patterns may be used to prevent habituation.

Stimulation parameters may be high frequency and low amplitude. Stimulation parameters may be used to create a sensation of touch, possibly in combination with a lower frequency and higher amplitude waveform that may also promote myoepithelial cell contraction. Multiple waveforms and settings may be available for mothers to select. Limitations to the settings (i.e. amplitude) may be used to prevent misuse and reduce risk to users.

Microcurrent stimulation (e.g., microamps, with a long 0.5 s pulse width) may also be used for sub-sensory stimulation. Microcurrents may stimulate ATP production, which may be useful for muscle contraction.

Stimulation may be controlled by a phone app. Use of a phone app may allow for use that is more discreet and convenient for users. The options of changing between different messaging patterns, frequencies, and amplitudes can be done through the app and on the device itself. Also, reminders, and pictures of the baby or favorite music may be added to the app for increased convenience.

The control system may be directly connected to the electrode array of inserts 304 via wires 314. In addition, a control system 316 may be connected to and control the electrode array inserts 304 via a wireless connection. For example, the control system 314 may be a cellphone having an application, sometimes called an “app,” that may control the operations of the electrode array of inserts 304.

FIGS. 4A and 4B show examples of two electrode stimulation configurations. As shown in FIG. 4A, a positive electrode 402 and a negative electrode 404 may be placed on opposite sides of a nipple 406 of a breast 408. While FIG. 4A may show the positive electrode 402 and the negative electrode 404 in the 9 o'clock and 3 o'clock positions about the nipple 406, the positive electrode 402 and the negative electrode 404 can be placed at any position about the nipple 406. For example, the positive electrode 402 and the negative electrodes 404 may be placed at the 12 o'clock and the 6 o'clock positions, respectively. Furthermore, while FIG. 4A shows the positive electrode 402 and the negative electrode 404 being positioned 180 degrees opposite one another, the positive electrode 402 and the negative electrode 404 may be positioned at any position about the nipple 406. For example, the positive electrode 402 and the negative electrode 404 may be placed at the 10 o'clock position and the 2 o'clock position, respectively.

FIG. 4B shows an example of circular electrodes. As shown in FIG. 4B, a positive electrode 412 may be proximate a nipple 416 and a negative electrode 414 may be located near a base 420 of the breast 418. The positions of the positive electrode 412 and the negative electrode 414 may be reversed. For instance, the negative electrode 414 may be proximate the nipple 416 and the positive electrode 412 may be located near the base 420 of the breast 418.

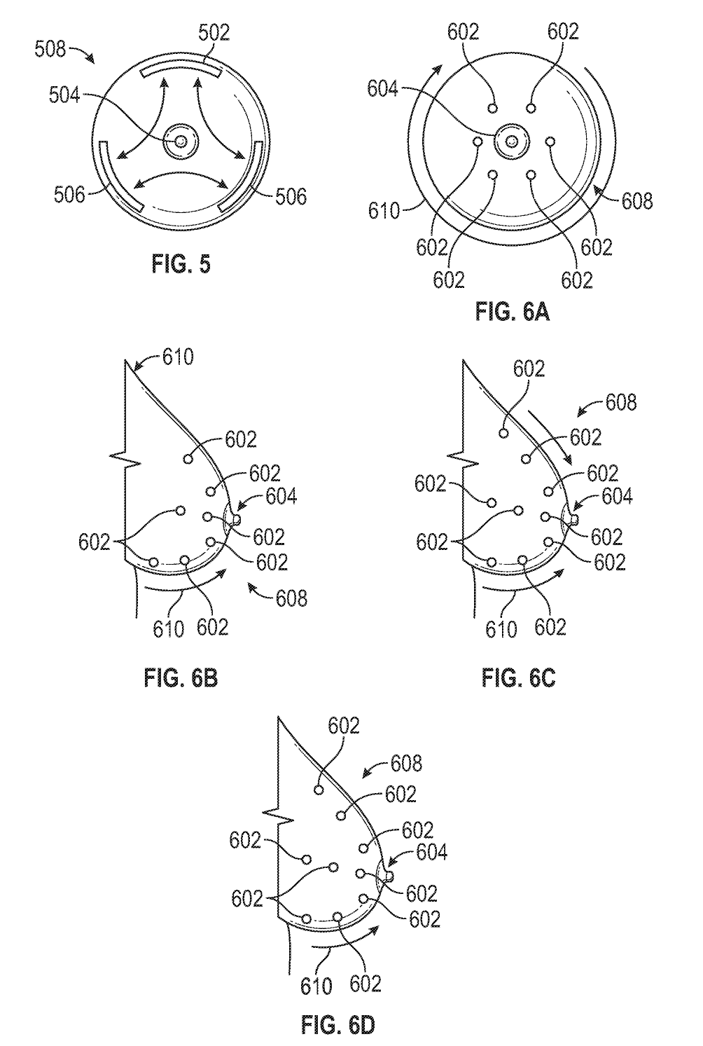

FIG. 5 shows an example of a three-electrode stimulation configuration. As shown in FIG. 5, a positive electrode 502 may be placed at a 12 o'clock position about a nipple 504 and two negative electrodes 506 may be placed at the 7 o'clock and 5 o'clock positions about the nipple 504. While FIG. 5 shows three electrodes, any number of electrodes could be used. For example, four electrodes could be used. The various electrodes can be placed at any location on the breast 508. In addition, the number of positive electrodes and the number of negative electrodes can be the same or can be different. For example, two positive electrodes could be used in conjunction with two negative electrodes. In addition, one positive electrode could be used in conjunction with three negative electrodes. Furthermore, while FIG. 5 shows the electrodes as being curved, the electrodes can be any shape. For example, the electrodes could be squares, circles, s-shaped, etc. For instance. FIGS. 6A-6D show that circular electrodes 602 can be placed in a pattern about the nipple 604.

As shown in FIGS. 6B-6D stimulation can be across the diameter of the breast 608 and proximal to distal towards the nipple 604 at the same time as represented by arrows 610. Opposing electrodes can be used to create stimulation across the breast and the stimulation can move as indicated by the arrows 610 to inner rings of electrodes. In addition, the stimulation can be rotational as shown in FIG. 6A. In other words, which electrodes are being excited back at the proximal end can be controlled to create a stimulation pattern that progresses from a distal end towards the nipple 604 in a circular pattern. For example, electrodes 602 could be placed to excite at 12 and 6 o'clock at the most proximal circumference and move distally towards the nipple 604, then excite at 3 and 9 o'clock back at the most proximal circumference and move distally towards the nipple 604.

The electrical stimulation can be done with direct current or high frequency alternating current. The stimulation can be applied at frequencies ranging from 0.2 Hz to 6 Hz. The frequency of the stimulation can be ramped up. For example, the initial frequency of stimulation may be 0.2 Hz and may ramp up to 6.0 Hz over a preset or random time period. For example, the frequency of stimulation may ramp up from 0.2 Hz to 3.0 Hz over a period of two minutes. The ramp up in frequency of stimulation may be constant or change over time. For example, the frequency of stimulation may ramp up in a linear, polynomial, logarithmic, etc. fashion.

The current used for stimulation may be constant or may vary. For example, the current may begin at 0.2 mA and may progress to a maximum current of 2.0 mA. The current may also decrease from 2.0 mA down to 0.1 mA. The stimulation may also be phased. For example, the stimulation provided by different pairs of electrodes may occur at different times or phases. For example, as shown in FIG. 6A six electrodes 602 may be placed about the nipple 604 and may be used to create stimulation in a circular pattern. For instance, the electrodes 602 may be activated in a circular pattern as indicated by numbers 1-6. In addition, as shown in FIG. 6B, the electrodes 602 may be activated to create a simulation that progress from the base 610 of the breast 608 and progresses towards the nipple 604.

The electrical stimulation may be in the form of vibration or other oscillations of tissue. The frequency of oscillation or vibration may be near the resonance frequency of a breast. The resonance frequency may be calculated as:

$$ω = \sqrt{\frac{k}{m}} ¶$$

where k is the elastic constant for breast tissue, approximately 3.25 kPa, and m is the mass of a breast, approximately 0.43-1.8 kg. Thus, the resonance frequency for an average breast may be between 87 and 42 Hz.

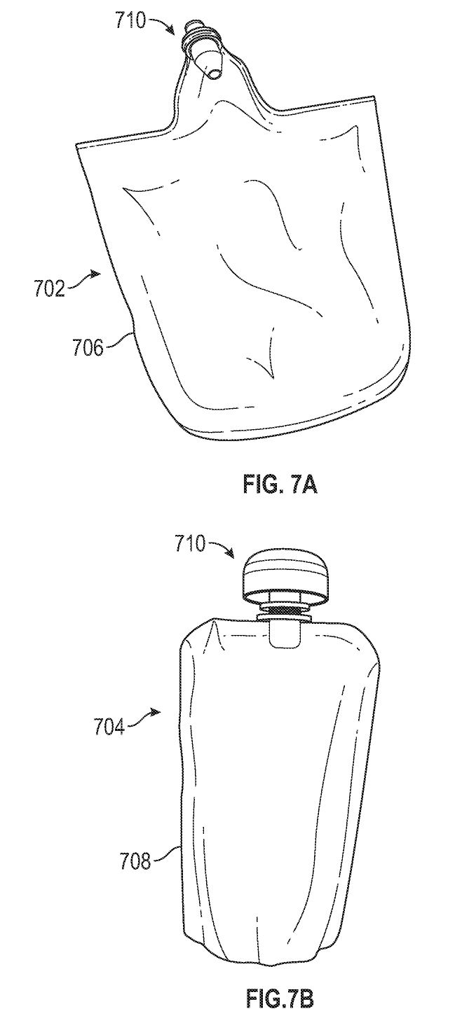

Embodiments may also include a breast seal and a connection to a flexible container as described herein. FIGS. 7A, 7B. 8A. 8B, 8C, and 8D, show collection devices 702, 704, and 802 consistent with embodiments disclosed herein. Each of the collection devices 702, 704, and 802 may include a container 706, 708, and 804, a connector 710 and 806, and a nipple interface 808.

The material of the containers 706, 708, and 804 may be flexible, non-toxic, placed in the freezer, and also thawed to room temperature. Thawing may be accomplished by placing a bag in warm water or placement in fridge without damaging or contaminating the breast milk.

The flexible containers 706, 708, and 804 may come in different sizes. A small container for freezing may hold 2-5 oz of breast milk and may have excess room for any expansion that may occur during freezing. The containers 708, 708, and 804 may also come in larger sizes for expressing milk over multiple sessions.

As shown in FIGS. 7A and 7B, the base of the containers 706 and 708 may have a bevel shape (e.g., rounded or rectangular) so that the container can stand upright. The material and bevel may be such that the containers 706 and 708 can stand up with minimal milk in it.

The containers 706, 708, and 804 may be transparent and may have measuring marks on it that may indicate how much milk may have been collected.

The containers 706, 708, and 804 may be sealed so milk leakage may be minimal when the containers 706, 708, and 804 are standing or lying on their side. The containers 706, 708, and 804 may have a one-way valve, or a check valve, so that when the containers 706, 708, and 804 are connected to the nipple interface 808 the valve may open and when disconnected may be closed. This valve, contained within the connectors 710 and 806 may also have a quick-connect feature so the containers 706, 708, and 804 may be easily connected/disconnected by the mother with one hand. A first portion of the one-way valve may be located proximate the electrode array and a second portion of the one-way valve may be connected to the containers 706, 708, and 804.

The containers 706, 708, and 804 may be disposable. The containers 706, 708, and 804 may be durable and safe enough to last 12 months in a freezer and with handling.

The containers 706, 708, and 804 may include ways to track the age of the milk. Example ways to track age include, but are not limited to, a space or numbers a mom can circle or a mechanical dial.

The containers 706, 708, and 804 may allow milk to flow freely from the mother's nipple into the containers 706, 708, and 804. In other words, milk may flow from the mother's nipple into the containers 706, 708, and 804 without the use of a pump or other artificial mechanism that induces flow. Factors such as gravity and smooth surfaces and a diameter of connection that does not impede flow may affect the ability of milk to flow freely from the mother's nipple into the containers 706, 708, and 804.

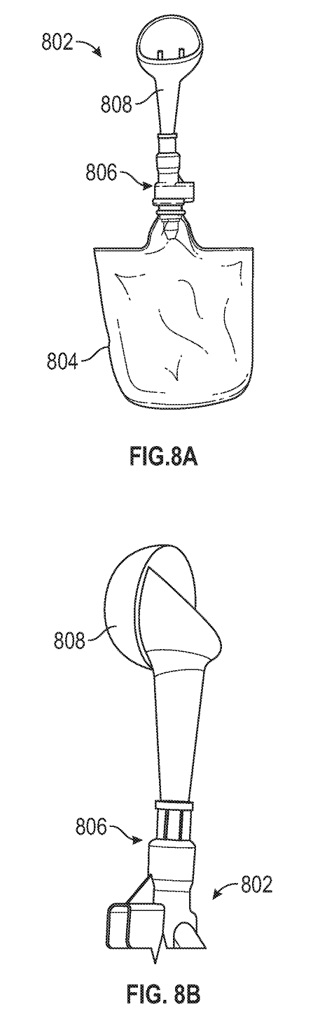

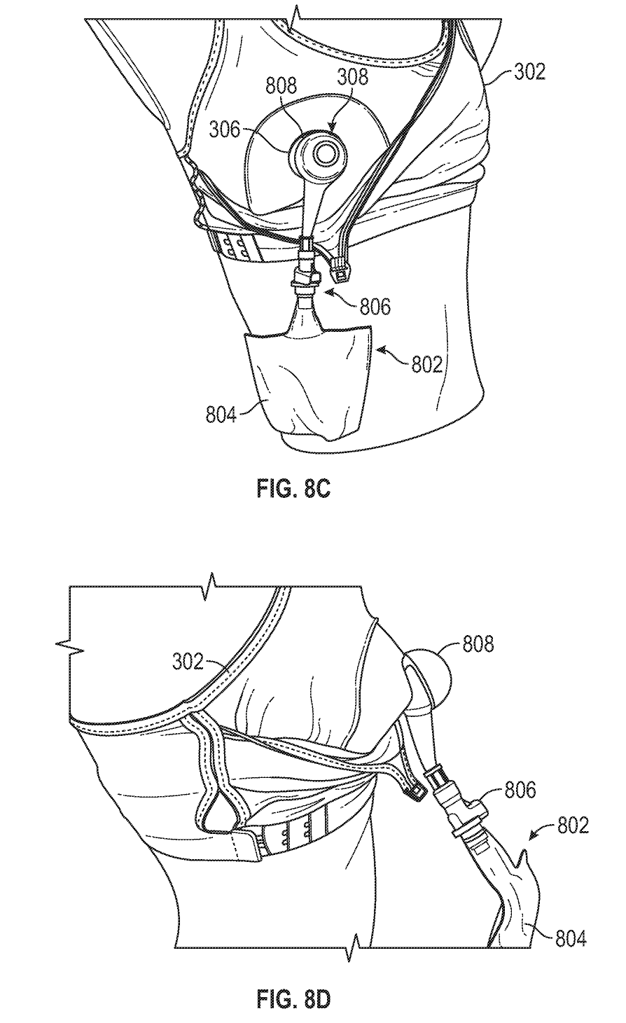

As shown in FIGS. 8A, 8B, 8C, and 8D, the containers 706, 708, and 804 may be designed to allow for the nipple interface 808 to be attached to a container 804 for direct consumption of breast milk. The nipple interface 808 can be any number of shapes. For examples, shape may be rounded to mimic the breast shape and the shape may constrict the flow of breast milk. For example, the shape may reduce flow so babies don't find the bottle easier than the breast.

The flexible containers 706, 708, and 804 can be supported by a garment or bra by clipping the container to the garment 302 as shown in FIGS. 8C and 8D. The garment 302 also could have a light mesh material to easily hold the container 706, 708, and 804.

The flexible container 706, 708, and 804 also may be used in lieu of a collection container for various breast pumps that may be available. The flexible containers 706, 708, and 804 may be attached to pumps with an adaptor for a particular brand and/or model pump. For example, threaded connections may be used for some brands and may not be used for other brands.

The flexible containers 706, 708, and 804 may include a diaphragm or similar system that may hinder milk from being sucked back into the breast pump and/or getting contaminated.

The containers 706, 708, and 804 may also include struts near the opening of the container to keep the container open. Stated another way, the struts near the opening of the containers 706, 708, and 804 may prevent the containers 706, 708, and 804 from closing due to suction or a vacuum that may be created within the containers 706, 708, and 804 and thus, preventing milk from entering the containers 706, 708, and 804.

The nipple interface 808 may have multiple rigid plastic pieces that connect the breast to the collection containers 706, 708, and 804. As shown in FIGS. 8C and 8D, a nipple of a mother may fit inside an aperture of the nipple interface 808 that may not irritate the sensitive nipple and may capture the milk being expressed. The connection may be semi-rigid, made of a soft comfortable material, and may be semi-circular or oval in shape. The connection component may be constructed of a flexible material filled with small silicone beads. The silicone beads may create a feeling similar to a baby's mouth on the breast.

The connection component may include one half of a one-way valve that may allow the flexible containers 706, 708, and 804 to be connected to it. The one-way valve may be closed when the container is not connected and open when the containers 706, 708, and 804 are connected to the breast. State another way, the valve may automatically shut when the containers 706, 708, and 804 are not connected to pump, breast, or otherwise being filled.

A streamline shape of this component may make it easy to clean and may prevent milk from getting caught in this section prior to reaching the collection container. An oleophobic (i.e., fat repelling) and hydrophobic (i.e., water repelling) coating also may be used to help with this.

Besides connecting to the collection containers 706, 708, and 804, the nipple interface 808 also may create a seal to the breast. One way of creating a seal with a breast may be to create a seal that may have a material like silicone that may be dome shaped and may include a hole in the middle to fit the shape of a nipple to be placed therein. This dome silicone shape may be inverted and may be reverted onto the breast to create the seal. The seal with the breast may also be created using an elastomeric tissue adhesive that may stick to the breast. The seal may be strong enough to support the weight of the component and the collection containers 702, 704, and 802.

In addition to, or supplemental to electrical stimulation, a garment or an insert may physically massage the breast in a way similar to hand expression techniques to promote milk expression. As discussed herein, the massage action may be created by a plurality of compartments that may be driven pneumatically or hydraulically. This device is connected to a flexible container that may collect expressed milk.

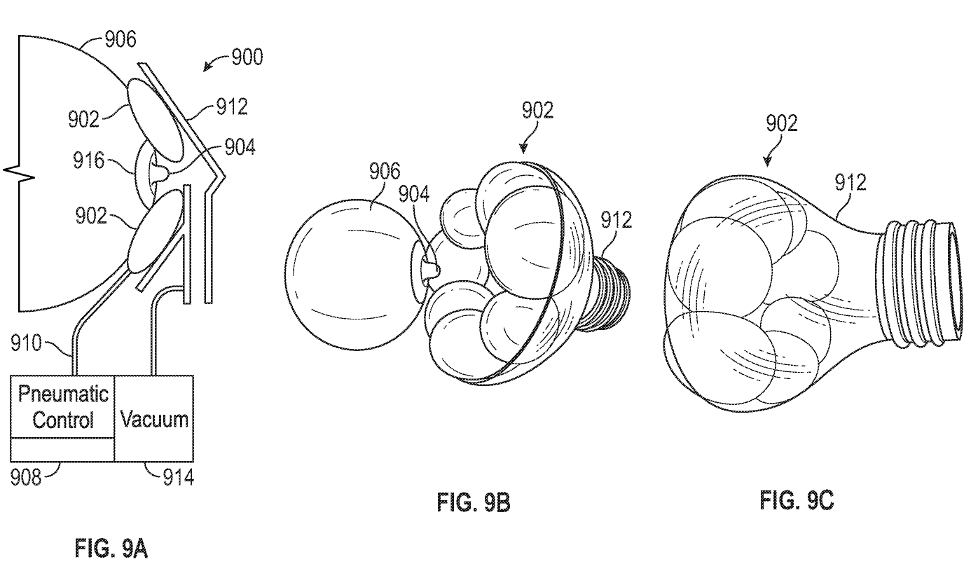

During a circumferential massage, the location of the inflating/deflating compartments may be more anterior or posterior. FIGS. 9A, 9B, and 9C show an example device 900 for circumferential massage. The device 900 may include compartments 902. The compartments 902 may be located approximately 1.5 inches away from a nipple 904. A fluid, such as air or a liquid, may be moved in and out of the compartments 902 for both comfort and massage effects. The pressure and frequency of massage can be adjusted this way as well.

The various compartments 902 may be inflated and deflated at various time intervals and in different patterns. For example, the various compartments 902 may be filled all at once or in opposing circumferential location (e.g., 6 and 12 o'clock or 3 and 9 o'clock). The compartments 902 may form rings from a posterior region of the breast 906, (i.e., the back of the breast) and may move towards the nipple 904. The user may be able to select amongst different massage types using a controller 908.

The controller 908 may be connected to the device 900 using a wire 910 or the controller 908 may communicate with the device 900 wirelessly using an application, sometimes called an “app,” on a cellphone or other handheld computing device. In addition, the controller 908 may be located on the device 900 itself.

The device 900 also may be fitted around the breast 906 and adjusted to different breast sizes. The fitting and adjusting may be accomplished via pneumatic or hydraulic filling of the compartments 902 of the device to the desired comfortable fit.

A soft material may be used to help fit the device 900 to the breast 906. The material may expand due to inflation on the side touching the breast 906 and being in contact with an exterior surface 912. The exterior surface 912 may be less elastic than the soft material and may be rigid. The exterior surface 912 may allow the compartments 902 to compress the breast 906 as opposed to the entire device expanding expand outwards.

Actuation of pneumatic or hydraulic control of the compartments 902 may be powered by a breast pump 914 or the pressure/vacuum from a separate pump. During use the collection of expressed milk can occur without the expressed milk passing through any pumps. In other words, the lactation devices disclosed herein can be configured such that expressed milk passes into collection containers without traveling through a pump that is part of the lactation devices.

A flat conformable force sensor, such as for example, a carbon nanotube force sensor, may be used as a safety to ensure no damage to the breast 906 is being incurred. For example, if the force on the breast 906 reaches a safety limit then the compression may automatically shut off and deflate the compartments 902.

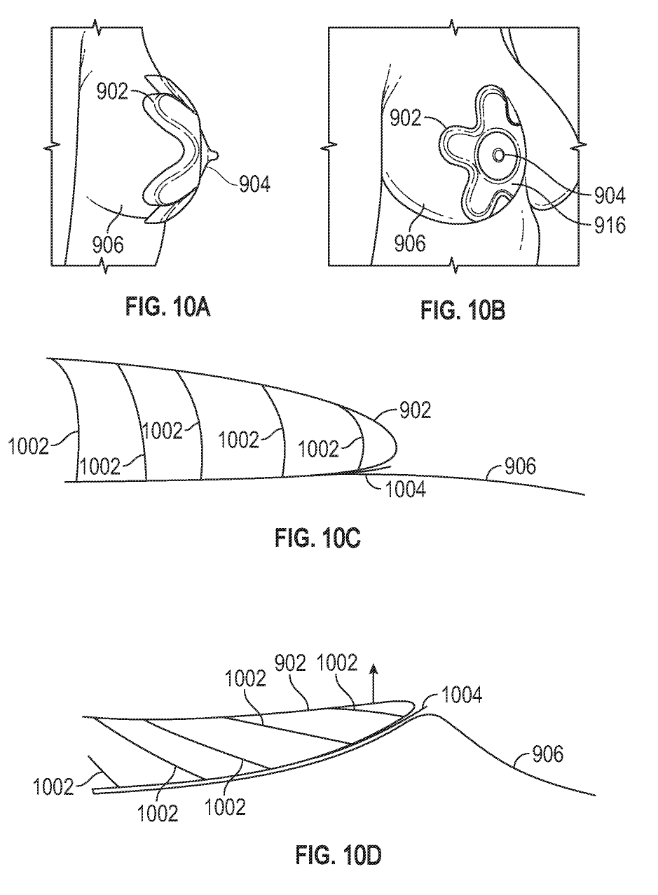

In addition, and as shown in FIGS. 10A, 10B, 10C, and 10D, the compartments 902 may have strain-limiting fibers 1002 that may give control and direction to cause actuation in a range of motions. The fibers 1002 may have different material properties, such as, but not limited to, material strength, modulus of elasticity, thickness, length, etc. For example, fibers 1002 of different material strengths may be used to cause the compartments 902 to bend and move inwards to compress the breast 906 similar to how fingers may massage a breast. The fibers 1002 may also allow for expansion of compartments 902 in a non-linear fashion. Furthermore, the fibers 1002 may also allow for a direction of expansion to be controlled. For example, using the fibers 1002, the compartments 902 may expand from a first end towards a second end. In addition, the fibers 1002 may be used to cause the compartments 902 to expand from a middle section outward to end sections. Moreover, the fibers 1002 can control bending of the compartments 902 as well. For example, the fibers 1002 can cause the compartments 902 to bend towards the breast 906 in a particular manner. For instance, the fibers 1002 can cause a center of the compartment 902 to bend towards the breast 906 while end sections of a compartments 902 deflect in a different or similar direction.

The fibers 1002 can be strands of a material such as, but not limited to, nylon, Kevlar, cotton, etc. In addition, the fibers 1002 can be a metallic material such as strands of aluminum, steel, etc. Furthermore, the fibers 1002 can be piezoelectric materials or shape memory alloys that can be actuated to apply pressure to the compartments 902, the breast, 906, etc.

The fibers 1002 may be internal to the compartments 902. The fibers 102 may be embedded within the material used to fabricate the compartments 902. The fibers 1002 may be external to the compartments 902. In addition, within a single compartment 902, or multiple compartments 902, some fibers 1002 may be internal, some fibers may be embedded, and other fibers 1002 may be external.

Embodiments may also include soft actuator technology that may use flexible materials that may include fluids. The fluids may move in and out of the compartments 902 to cause actuation changes. Rigid, semi rigid, and strain-limiting elements and fibers, such as shown in FIGS. 10A-10D may also provide control and range of motion.

Embodiments may include, but are not limited to, soft actuators that may be powered by vacuum from a breast pump: soft actuators that may be powered by pressure/vacuum from a separate fluid pump; and soft actuators that may be powered by heating and cooling of elements that may be located within cavities of the actuators that may expand/contract the fluids within the actuators.

Embodiments may also include flexible materials used in conjunction with nitinol wire (which fibers 1002 may be constructed of) that may be used with heating and cooling elements to actuate the materials.

Embodiments may also include a self-adhesive backing 1004 that may reduce size and visibility by eliminating clasps, harnesses, and other attachment features such as shown in FIGS. 10A-10D. The adhesive backing 1004 also may facilitate application and hands-free operation. The adhesive backing 1004 also may provide strain limitation to the actuator and may limit stretching and pinching of breast tissues. The adhesive backing 1004 may be a substrate material or other membrane material. For example, the adhesive backing 1004 may be impregnated with a disinfectant or other topical ointments to provide treatment to breast or other skin tissue.

Low-friction coatings also may be used to simulate the feel of a baby's mouth. Low-friction coating may include, but are not limited to, hydrophilic coatings, hydrophilic hydrogel coatings that may be rehydrated by the wetting or the milk being pumped, and coatings in conjunction with soft actuators that may stimulate the nipple 904 and aureole 916 through slick friction and compression.

Fluid bladders (e.g., the compartments 902), cables in tension (e.g., the fibers 1002), etc., may be used to achieve mechanical stimulation. Mechanical stimulation may be in the form of vibration or other oscillations of tissue. The frequency of oscillation or vibration may be near the resonance frequency of a breast as described above with regards to electrical stimulation.

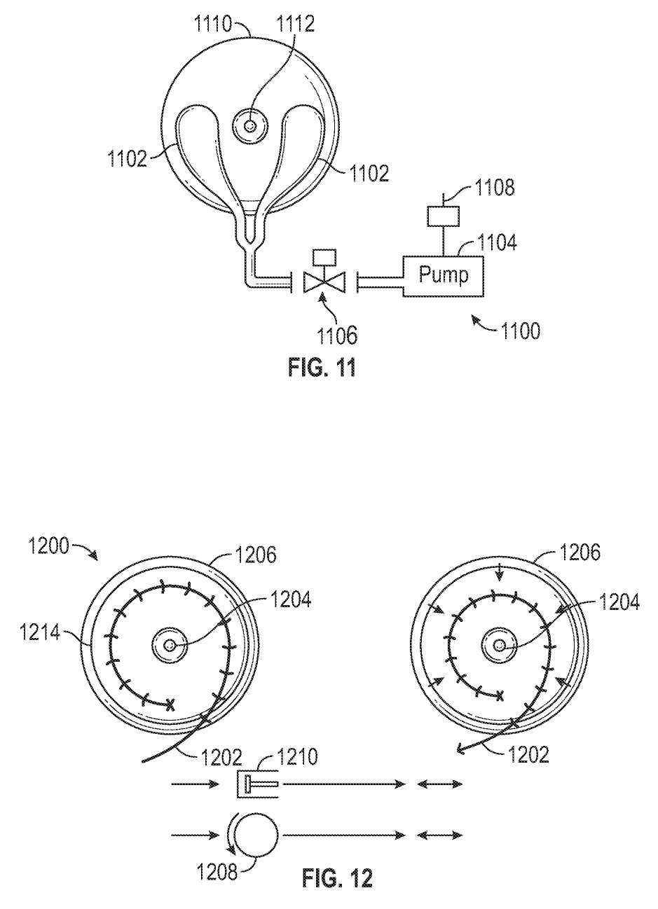

FIG. 11 shows an example of a fluid bladder system 1100 for mechanical stimulation. FIG. 11 shows bladders 1102 that may be operated by a pump 1104 in conjunction with a valve 1106. The bladders 1102 may be attached directly to a bra or a bra may include pouches into which the bladders 1102 can be installed. The valve 1106 may be a solenoid valve that may be controlled by a controller 1108. More than one valve may be used. For example, each bladder 1102 may have its own valve to control inflation and deflation of the bladder 1102. The pump 1104 may be a positive pressure pump or a vacuum pump. The pump 1104 may be a hydraulic pump or a pneumatic pump. The use of pumps and valves may create pulses that can stimulate the breast 1110 proximate the nipple 1112. The valves and pumps may operate at the same or different frequencies from one another. For example, the pumps may cycle at a first frequency and the valves may operate at a second frequency in order to simulate a suckling child and/or otherwise stimulate the breast.

FIG. 12 shows a cable configuration 1200 for mechanical stimulation. As shown in FIG. 12, a cable 1202 can be attached to a bra in a pattern that surrounds a nipple 1204 of a breast 1206. The cable 1202 can be directly attached to the bra or may pass through a sleeve of the bra. A motor 1208 or air cylinder 1210 may be attached to a free end of the cable 1202. During operation, the motor 1208 or air cylinder 1210 may cause the cable 1202 to tighten and relax at a constant or irregular frequency.

Example lactation assistive devices can include the cable 1202 and a controller (as described above). The cable 1202 can be coupled to a garment 1214 (e.g., a bra) as described herein. The cable 1202 can be located at least partially proximate the nipple 1204 of the breast 1206 when the garment 1214 is worn about the breast 1206. For instance, the cable 1202 can be attached to the garment 1214 in a spiral pattern that centers around the nipple 1204. The controller can be configured to control movement of the cable 1202. During use, movement of the cable 1202 can cause mechanical stimulation of the breast 1206. In addition, a plurality of fibers (such as fibers 1002) can be connected to the garment 1214 proximate the cable 1202 and arranged to control movement of the cable 1202. While the device 1200 may be described with integration directed toward a bra, the device 1200 can be standalone device that can be used with various garment types. For example, the device 1200 can be a portable unit that can be transferred between garments.

The device 1200 can further include an aperture or other connector that can located proximate the nipple 1204 of the breast 1206. The port can include a fluid orifice that can be configured to allow expressed milk to pass through the port for collection.

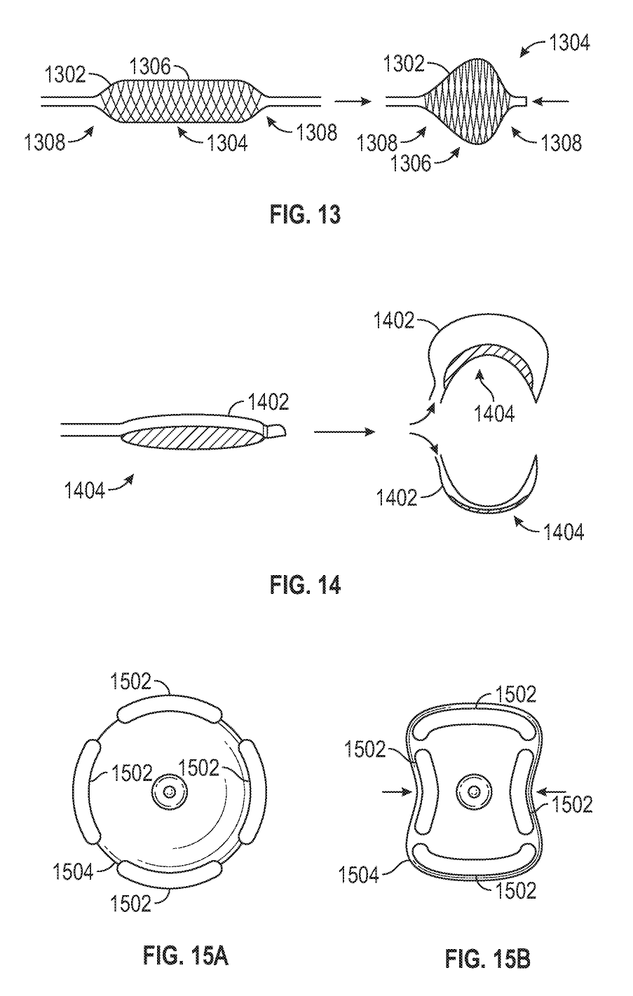

As discussed herein, fibers may be used to control the inflation and deflation of the various bladders 902. As shown in FIG. 13, a bladder 1302 may have fibers 1304 that restrict the inflation to cause a center portion 1306 of the bladder 1302 to expand more than end portions 1308 of a bladder 1302. The fibers 1304 may also restrict the expansion rate of the bladder 1302. For example, the fibers 1304 may allow the center 1306 of the bladder to expand at a faster rate than the ends 1308 of the bladder 1302.

As shown in FIG. 14, a bladder 1402 may have fibers 1404 that partially cover the bladder 1402. The fibers 1404 may cause the bladder 1402 to expand in different directions. For example, the fibers 1404 may be located on an underside of the bladder 1402 and may cause the bladder 1402 to curl around the breast upon inflation. The fibers 1404 may be located on an outer surface of the bladder 1402 and may cause a center portion of the bladder 1402 to press into the breast upon inflation.

FIGS. 15A and 15B show an example of multiple bladders 1502 used to provide mechanical stimulation. The various bladders 1502 can be position around a base of breast 1504. Upon actuation, the various bladders 1502 can provide uniform compression of the breast 1504 in a circumferential manner. The various bladders 1502 can be inflated in a non-uniform manner. The non-uniform inflation of the bladders 1502 may stimulate the breast 1504 in a pattern. For example, the non-uniform inflation of the bladders may stimulate the breast in a circular manner.

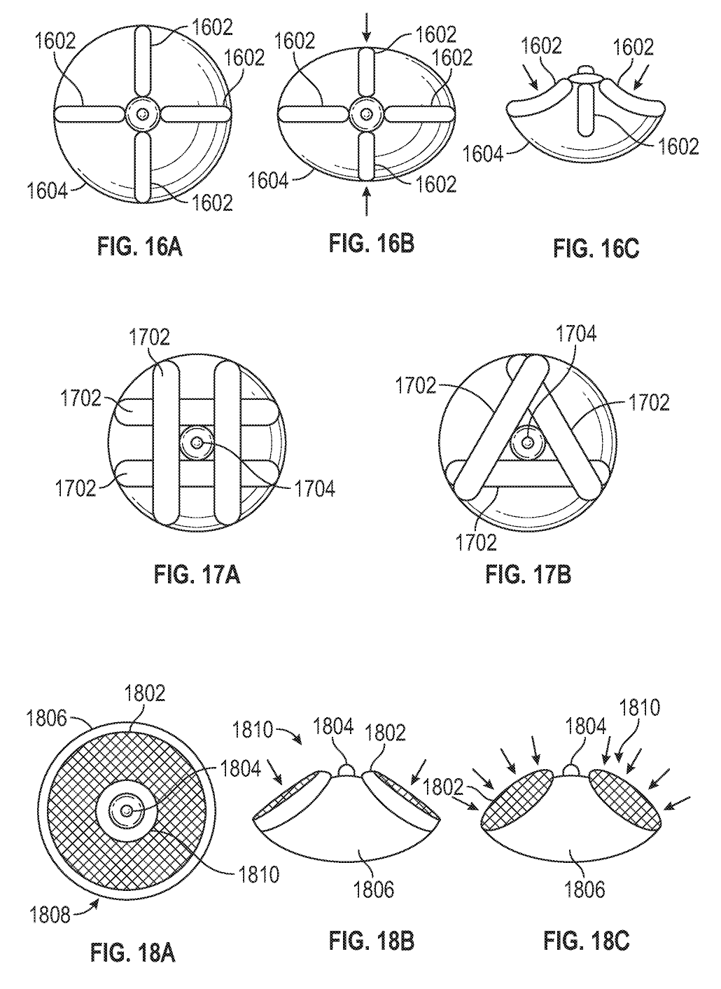

FIGS. 16A, 16B, and 16C show an example of multiple bladders 1602 arranged in a radial pattern around a breast 1604. The various bladders 1602 can provide mechanical stimulation in a uniform pattern or in a random pattern. For example, the bladders 1602 may allow for uniform compression of the breast 1604 in an inward manner as shown in FIGS. 16A-16C. The various bladders 1602 may inflate at separate time intervals to allow for a circular stimulation of the breast 1604.

FIGS. 17A and 17B show examples of multiple bladders 1702 arranged in cross configurations. As shown in FIG. 17A, the bladders 1702 may be crossed in a square or rectangular manner around a nipple 1704. FIG. 17B shows bladders 1702 arranged in a triangular pattern around the nipple 1704. The various bladders 1702 may have fibers to control inflation as described herein. In addition, the various bladders 1702 may inflate and deflate in unison, at different times, and at different rates.

FIGS. 18A-18C show an example of a circular bladder 1802. The bladder 1802 may have compartments that allow the bladder 1802 to be inflated in a circular manner around the nipple 1804 and breast 1806. The bladder 1802 may include fibers 1808 that control the rate and direction of inflation. In addition, the bladder 1802 may include an opening 1810 that centers about the nipple 1804 to allow for expression of milk.

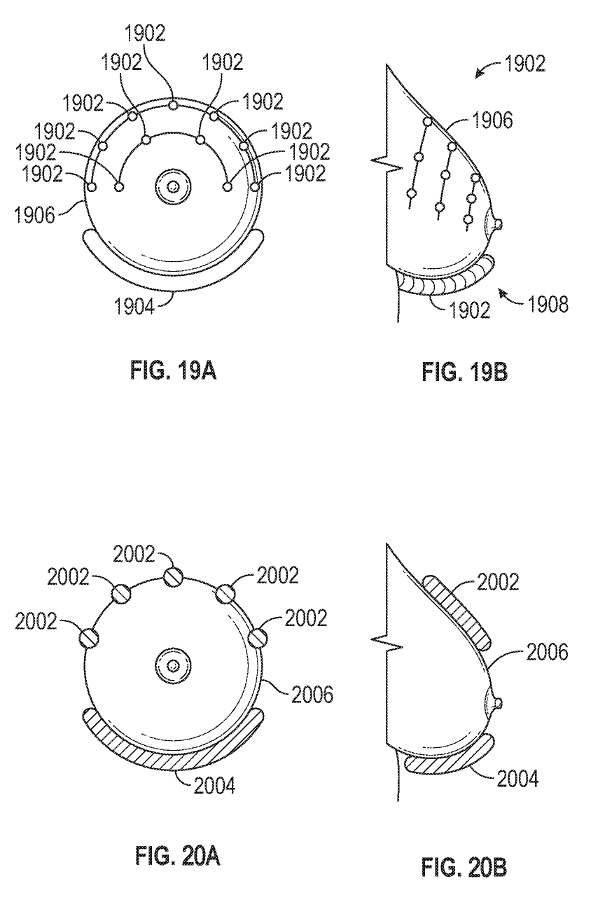

As shown in FIGS. 19A and 19B, solid members 1902 may be used in conjunction with a bladder 1904 to stimulate a breast 1906. For example, solid members 1902, such as beads, tubes, or other members, may be placed on or within a bra. A bladder 1904 or bladders within the bra may inflate and cause the breast 1906 to be pressed into the solid members 1902. The solid members 1902 may cause a kneading type stimulation of the breast 1906. A single bladder 1904 may have fibers 1908 as described herein to control inflation of the bladder 1902 and thereby control stimulation of the breast 1906 with the solid members 1902. In addition, multiple bladders may be inflated and deflated at different times or at different rates to control stimulation of the breast 1906.

As shown in FIGS. 20A and 20B, articulating members 2002 may be used in conjunction with a stationary bladder 2004 to stimulate the breast 2006. For example, articulating members 2002 may be located at various locations about the breast 2006. In addition, the stationary bladders 2004 may be located at a location or locations about the breast 2006. During use, the articulation member 2002 may apply pressure to the breast 2006 at various locations. The pressure may be applied uniformly, at intervals, in predetermined patterns, and at random patterns. The stationary bladder 2004 can maintain a constant force on the breast 2006 during stimulation. The stimulation can be caused as the articulation bladders 2002 inflate and cause the breast 2006 to be pressed into the stationary bladder 2004. In addition, the force applied by the stationary bladder 2004 can cause the articulating bladders 2002 to press into the breast 2006 for stimulation.

### Examples

Example 1 is a lactation assistive device comprising: an electrode array sized to fit within a garment worn about a breast, the electrode array defining an opening to be located proximate a nipple of the breast when the garment is worn; and a controller configured to control electrical stimulation of the breast by the electrode array.

In Example 2, the subject matter of Example 1 optionally includes a container having a passage to be located proximate the nipple of the breast, the container for collecting milk expressed from the breast.

In Example 3, the subject matter of Example 2 optionally includes wherein the container includes a first portion of a check valve and the electrode array includes a second portion of the check valve.

In Example 4, the subject matter of any one or more of Examples 1-3 optionally include wherein the controller is programmed to deliver uniform excitation to the electrode array.

In Example 5, the subject matter of any one or more of Examples 1-4 optionally include wherein the controller is programmed to deliver excitation to the electrode array that progresses inward from a perimeter of the electrode array toward a center of the electrode array, the center of the electrode array located proximate the nipple.

In Example 6, the subject matter of any one or more of Examples 1-5 optionally include wherein the controller is programmed to deliver excitation to the electrode array at opposing points along a diameter.

In Example 7, the subject matter of any one or more of Examples 1-6 optionally include wherein the controller is programmed to provide high frequency electrical stimulation and low pulse electrical stimulation.

In Example 8, the subject matter of any one or more of Examples 1-7 optionally include wherein the controller is programmed to provide electrical stimulation that simulates a sensation of touch.

In Example 9, the subject matter of any one or more of Examples 1-8 optionally include wherein the controller is a handheld computing device.

In Example 10, the subject matter of any one or more of Examples 1-9 optionally include wherein the controller is programmed to provide high frequency and low amplitude electrical stimulation.

Example 11 is a lactation assistive device comprising: a plurality of compartments sized to receive a fluid, the plurality of compartments arranged to form an opening to be located proximate a nipple of a breast when the plurality of compartments are worn within a garment about the breast; fibers connected to each of the plurality of compartments, the fibers arranged to control expansion of the plurality of compartments; and a controller configured to control movement of the fluid within the plurality of compartments in order to mechanically stimulate the breast.

In Example 12, the subject matter of Example 11 optionally includes wherein the fluid is a gas.

In Example 13, the subject matter of any one or more of Examples 11-12 optionally include wherein the fluid is a liquid.

In Example 14, the subject matter of any one or more of Examples 11-13 optionally include a rigid surface to be located between the garment and the plurality of compartments when worn.

In Example 15, the subject matter of any one or more of Examples 11-14 optionally include a flexible container having a passage to be located proximate the nipple of the breast, the flexible container for collecting milk expressed from the breast.

In Example 16, the subject matter of any one or more of Examples 11-15 optionally include wherein the controller is programmed to deliver the fluid circumferentially across the breast.

In Example 17, the subject matter of any one or more of Examples 11-16 optionally include wherein the controller is programmed to deliver the fluid radially across the breast.

In Example 18, the subject matter of any one or more of Examples 11-17 optionally include wherein the controller is a handheld computer.

Example 19 is a lactation assistive device comprising: a cable coupled to a garment, the cable to be located at least partially proximate a nipple of a breast when the garment is worn about the breast; and a controller configured to control movement of the cable, wherein movement of the cable causes mechanical stimulation of the breast.

In Example 20, the subject matter of Example 19 optionally includes a port located proximate the nipple of the breast, the port including a fluid orifice configured to allow expressed milk to pass through the port.

In Example 21, the subject matter of any one or more of Examples 19-20 optionally include wherein the controller is a handheld computer.

In Example 22, the subject matter of any one or more of Examples 19-21 optionally include wherein the cable is couple to the garment in a circular pattern centered about the nipple.

In Example 23, the subject matter of any one or more of Examples 19-22 optionally include a plurality of fibers connected to the garment proximate the cable, the fibers arranged to control movement of the cable.

Example 24 is a lactation assistive device comprising: a garment including a plurality of compartments sized to receive a fluid, the plurality of compartments arranged to form an opening to be located proximate a nipple of a breast when the plurality of compartments are worn within the garment about the breast; an electrode array sized to fit within the garment worn about the breast, the electrode array defining an opening to be located proximate the nipple of the breast when the garment is worn; and a controller configured to: control electrical stimulation of the breast by the electrode array, and control movement of the fluid within the plurality of compartments in order to mechanically stimulate the breast.

In Example 25, the subject matter of Example 24 optionally includes fibers connected to each of the plurality of compartments, the fibers arranged to control expansion of the plurality of compartments.

In Example 26, the subject matter of any one or more of Examples 24-25 optionally include a rigid surface to be located between the garment and the plurality of compartments when worn.

It will be readily understood to those skilled in the art that various other changes in the details, material and arrangements of the parts and method stages which have been described and illustrated in order to explain the nature of the inventive subject matter may be made without departing from the principles and scope of the inventive subject matter as expressed in the subjoined claims.

1. A lactation assistive device comprising:
an electrode array sized to fit within a garment worn about a breast, the electrode array defining an opening to be located proximate a nipple of the breast when the garment is worn; and
a controller configured to control electrical stimulation of the breast by the electrode array.

2. The lactation assistive device of claim 1, further comprising a container having a passage to be located proximate the nipple of the breast, the container for collecting milk expressed from the breast.

3. The lactation assistive device of claim 2, wherein the container includes a first portion of a check valve and the electrode array includes a second portion of the check valve.

4. The lactation assistive device of claim 1, wherein the controller is programmed to deliver uniform excitation to the electrode array.

5. The lactation assistive device of claim 1, wherein the controller is programmed to deliver excitation to the electrode array that progresses inward from a perimeter of the electrode array toward a center of the electrode array, the center of the electrode array located proximate the nipple of the breast.

6. The lactation assistive device of claim 1, wherein the controller is programmed to deliver excitation to the electrode array at opposing points along a diameter.

7. The lactation assistive device of claim 1, wherein the controller is programmed to provide high frequency electrical stimulation and low pulse electrical stimulation.

8. The lactation assistive device of claim 1, wherein the controller is programmed to provide electrical stimulation that simulates a sensation of touch.

9. The lactation assistive device of claim 1, wherein the controller is a handheld computing device.

10. A lactation assistive device comprising:
a plurality of compartments sized to receive a fluid, the plurality of compartments arranged to form an opening to be located proximate a nipple of a breast when the plurality of compartments are worn within a garment about the breast;
fibers connected to each of the plurality of compartments, the fibers arranged to control expansion of the plurality of compartments; and
a controller configured to control movement of the fluid within the plurality of compartments in order to mechanically stimulate the breast.

11. The lactation assistive device of claim 10, wherein the fluid is a gas.

12. The lactation assistive device of claim 10, wherein the fluid is a liquid.

13. The lactation assistive device of claim 10, further comprising a rigid surface to be located between the garment and the plurality of compartments when worn.

14. The lactation assistive device of claim 10, further comprising a flexible container having a passage fluidly connecting the opening and an interior of the flexible container, the flexible container for collecting milk expressed from the breast.

15. The lactation assistive device of claim 10, wherein the controller is programmed to deliver the fluid circumferentially across the breast.

16. The lactation assistive device of claim 10, wherein the controller is programmed to deliver the fluid radially across the breast.

17. The lactation assistive device of claim 10, wherein the controller is a handheld computer.

18. A lactation assistive device comprising:
a garment including a plurality of compartments sized to receive a fluid, the plurality of compartments arranged to form an opening to be located proximate a nipple of a breast when the plurality of compartments are worn within the garment about the breast;
an electrode array sized to fit within the garment worn about the breast, the electrode array defining an opening to be located proximate the nipple of the breast when the garment is worn; and
a controller configured to:
control electrical stimulation of the breast by the electrode array, and
control movement of the fluid within the plurality of compartments in order to mechanically stimulate the breast.

19. The lactation assistive device of claim 18, further comprising fibers connected to each of the plurality of compartments, the fibers arranged to control expansion of the plurality of compartments.

20. The lactation assistive device of claim 18, further comprising a rigid surface to be located between the garment and the plurality of compartments when worn.
